(19) United States
(12) Patent Application Publication
(10) Pub. No.: US 2009/0291167 A1
(43) Pub. Date: Nov. 26, 2009

(54) Composition and Process for Flavouring Dairy Products, Strain of Lactic Acid Bacterium, Use of Said Composition or Strain

(75) Inventors: Annie Mornet, Mondion (FR); Jonathan Goodwins, Abilly (FR)

Correspondence Address: HOWREY LLP - East, C/O IP DOCKETING DEPARTMENT, 2941 FAIRVIEW PARK DR, SUITE 200, FALLS CHURCH, VA, 22042-2924, US

(73) Assignee: DANISCO A/S, Copenhagen (DK)

(21) Appl. No.: 12/161,386

(22) PCT Filed: Jan. 17, 2007
(86) PCT No.: PCT/FR07/00085

§ 371 Date: Jul. 13, 2009

(30) Foreign Application Priority Data
Jan. 20, 2006 (FR) 0600546

(51) Int. Cl. A23C 9/12; C12N 1/20; A23L 1/28; A23C 19/00

(52) U.S. Cl. 426/43; 435/252.9; 426/61

(57) ABSTRACT

Composition for flavouring dairy products, in particular cheeses, comprising at least one combination of microorganisms, said combination comprising at least one microorganism of lytic type and at least one ripening agent, flavouring process, use of said compositions or strains.

The present invention relates to the dairy industry, in particular the manufacture of dairy products, and more particularly the manufacture of cheeses, in particular the manufacture of uncooked pressed cheese.

The manufacture of dairy products requires the use of various microorganisms having specific roles in food technology.

In particular, the lactic acid bacteria are used in the acidification of milk, which causes fermentation of milk to produce curds. The lactic acid bacteria are added to the milk to be fermented in the form of ferments, also called acidification ferments, starter cultures or starters.

Other microorganisms are in their turn used for the ripening of cheeses. In this case they are called ripening flora, or ripening agents, or non-starters. These microorganisms make it possible to convert milk curds to a partially lipolyzed, proteolyzed product, enriched in aromatic compounds. The aromatic compounds are the compounds which develop the flavour of the cheese. They consist for example of amino acids, fatty acids, aromatic peptides or volatile compounds such as esters, volatile ketonic or sulphur-containing derivatives etc. Flavour means, in the present document, all of the olfactory, gustatory and trigeminal sensations perceived in the course of tasting.

The ripening agents currently used are bacteria, for example bacteria of the genera Arthrobacter, Corynebacterium, Lactobacillus, Lactococcus, Leuconostoc, Micrococcus, Pediococcus, Propionibacterium, Staphylococcus and Streptococcus. They can also be yeasts or moulds, such as for example the microorganisms of the genera Candida, Debaryomyces, Geotrichum, Kluyveromyces, Rhodotorula, Saccharomyces or Penicillium.

Ripening is of great importance for imparting flavour to a dairy product.

The addition of traditional ripening agents to the acidification ferments is not enough in certain cases to develop the desired flavour in a dairy product. Indeed, the traditional ripening agents do not provide sufficient flavouring of the dairy product, in particular when the ripening time is short or reduced. In this case, the sought aromatic profile is not obtained, and other undesirable flavours, for example bitterness, may appear.

In order to meet the requirements of consumers and manufacturers, it has become essential to find new microorganisms capable of ripening and flavouring dairy products.

Moreover, the use of microorganisms of the genus Lactobacillus of the non-lytic type is known.

In addition, the use of ripening agents of the genus Brevibacterium for the flavouring of cheese is known.

However, the combined use of these two strains does not make it possible to achieve ripening and flavouring of dairy products capable of meeting the aforementioned requirements.

The purpose of the invention is to propose a novel means of flavouring dairy products which offers many qualities and makes it possible to develop the desired flavour for dairy products, in a significant, quick and permanent manner.

To this end, a subject of the invention is a composition for flavouring dairy products, in particular cheeses, comprising at least one combination of microorganisms, said combination comprising at least one microorganism of lytic type and at least one ripening agent.

“Microorganism of lytic type” means any acidification or ripening bacterium having enhanced enzymatic properties. In particular, in the present case, these are microorganisms which release, by autolysis, after a conventional ripening time, 40% or more of their intracellular proteolytic enzymes into their surrounding medium when used under conventional conditions (i.e. under conditions of physical stresses, salinity and pH similar to those present in a dairy product).

Typically, in order to measure the percentage of intracellular proteolytic enzymes released into the surrounding medium by autolysis, a person skilled in the art can compare the intracellular activity of the dipeptidase PepD from a specific quantity of microorganisms and compare it with the enzymatic activity of the dipeptidase PepD released by the same quantity of microorganisms into the surrounding medium after 24 hours at 40° C. in a medium buffered to a pH of 5.8 with 0.1 M potassium phosphate buffer.

The microorganisms of lytic type are for example the acidification or ripening lytic lactic acid bacteria, for example of the genera Lactococcus, Lactobacillus, Pediococcus and Leuconostoc of the lytic type.

Ripening agents are for example bacteria and in particular the bacteria of the genera Arthrobacter, Corynebacterium, Lactobacillus, Lactococcus, Leuconostoc, Micrococcus, Pediococcus, Propionibacterium, Staphylococcus and Streptococcus. They can also be yeasts or moulds, such as for example the microorganisms of the genera Candida, Debaryomyces, Geotrichum, Kluyveromyces, Rhodotorula, Saccharomyces or Penicillium.

In fact, the use of microorganisms of lytic type in combination with at least one ripening agent for the ripening of dairy products makes it possible on the one hand to enhance, i.e. to increase or reinforce, the development of flavour relative to the use of microorganisms of conventional ripening flora.

Typically, the ratio of the number of microorganisms of lytic type to ripening agents can be comprised between 30/70 and 70/30, and preferably the ratio can be comprised between 40/60 and 60/40.

This synergy is manifested not only by obtaining a significantly greater quantity of conventional flavouring compounds in the cheese such as those of the diacetyl, 2-butanone, acetoin, DMDS (dimethyl disulphide), 1-octen-3-ol, DMTS (dimethyl trisulphide) and butyric acid type.

The action of microorganisms of lytic type in combination with a ripening agent also makes it possible, by synergy with said ripening agent, to promote the development of the flavour by said ripening agent. It allows Brevibacterium linens in particular to produce a larger amount of sulphur-containing compounds.

Moreover, the use of microorganisms of lytic type for the ripening of dairy products makes it possible to reduce the proportion of compounds responsible for flavour of the bitter type.

Finally, the use of microorganisms of lytic type in combination with a ripening agent allows the production of small (under 1 kDa), hydrophilic peptides, which are not produced when the different strains are cultivated independently. This synergy therefore allows the production of different peptides which impart novel tastes (for example of the meat stock, umami, savoury type) to the finished dairy product.

Another advantage of the present invention is that the microorganism of lytic type can be used together with bacteria used for the acidification of dairy products, i.e. with acidification ferments (for example of the type Lactococcus lactis and/or Streptococcus thermophilus).

Advantageously, the microorganism of lytic type is of the genus Lactococcus or of the genus Lactobacillus, in particular of the species Lactobacillus helveticus or Lactobacillus casei.

Advantageously, said ripening agent is a microorganism of the genus Arthrobacter or Brevibacterium, in particular of the genus Brevibacterium linens.

The use of the combination of microorganisms of the genus Lactobacillus helveticus of lytic type and of microorganisms of the genus Brevibacterium makes it possible to reinforce the development of flavour by the microorganisms of the genus Brevibacterium.

In a preferred embodiment, said microorganism of lytic type is the strain Lactobacillus helveticus LbH 210 deposited in the name of Danisco France SAS, 20 rue de Brunel, 75017 Paris, at the CNCM (Collection Nationale de Culture de Microorganismes) on 20 Jan. 2006 under number CNCM I-3554.

In another advantageous embodiment, said ripening agent is the strain Brevibacterium 3306 deposited in the name of Danisco France SAS, 20 rue de Brunel, 75017 Paris, at the CNCM on 20 Jan. 2006 under number CNCM I-3556.

In another advantageous embodiment, said ripening agent is the strain Brevibacterium 3383 deposited in the name of Danisco France SAS, 20 rue de Brunel, 75017 Paris, at the CNCM on 20 Jan. 2006 under number CNCM I-3555.

In a particular embodiment, said microorganisms of lytic type and said ripening agents are in a mixture or in separate form.

Advantageously, said microorganisms of lytic type and said ripening agents are in the form of a lyophilized or frozen mixture of strains, or in the form of strains packed separately.

The present invention also relates to a strain Lactobacillus helveticus LbH 210 deposited in the name of Danisco France SAS, 20 rue de Brunel, 75017 Paris, at the CNCM on 20 Jan. 2006 under number CNCM I-3554.

This strain makes it possible to enhance the flavour developed by the ripening agents of a dairy product.

Another subject of the present invention is a method for flavouring a dairy product, in particular an uncooked pressed cheese, comprising the addition of a flavouring composition in milk.

The addition of a flavouring composition in milk provides the method for flavouring a dairy product with a time saving as it makes it possible to reduce the ripening time. In addition, it also makes it possible to reduce the formation of compounds which are responsible for undesirable bitter flavours, which limits the risks of withdrawal from sale of dairy products not meeting consumers' requirements.

In an advantageous embodiment, the concentration of microorganisms of lytic type and ripening agents is between 10⁴ and 10⁸ CFU/g of dairy product, preferably approximately 10⁶ CFU/g of dairy product. Typically, the ratio of the number of microorganisms of lytic type to ripening agents can be comprised between 30/70 and 70/30, preferably the ratio can be comprised between 40/60 and 60/40.

In a particularly advantageous embodiment of the method, the microorganisms of lytic type and the ripening agents are added at a rate of 10⁶ to 10⁹ CFU per litre of milk, preferably approximately 10⁸ CFU per litre of milk.

Preferably, the milk is of animal origin.

Finally, the invention relates to the use of a composition comprising a combination of microorganisms, said combination comprising at least one microorganism of lytic type and at least one ripening agent for flavouring dairy products, in particular cheeses.

The use of such a composition for flavouring dairy products makes it possible to obtain a dairy product which is significantly matured, i.e. significantly matured relative to a conventional method of flavouring. In addition, this use makes it possible to obtain a matured dairy product which has no unpleasant tastes or unpleasant odours.

In an advantageous embodiment, said at least one microorganism of lytic type used is preferably of the genus Lactobacillus helveticus of lytic type, and said ripening agent used is preferably of the genus Brevibacterium.

Advantageously, the dairy product is selected from soft cheese, uncooked pressed cheese, cooked cheese, fromage frais, veined cheese, cheese spreads and “analogue cheese” (based on casein powder and/or whey proteins), enzyme-modified cheese, cottage cheese, as well as yoghurt, ripened cream, milk drinks, fermented milk, a dairy product retentate, a hydrolysate of vegetable proteins, for example soya, or a baby milk.

Even more advantageously, the dairy product is of the uncooked pressed cheese type, and preferably of the Gouda or Cheddar type.

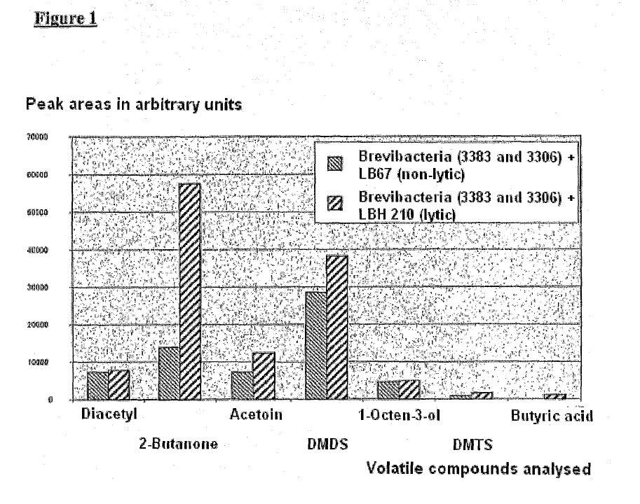

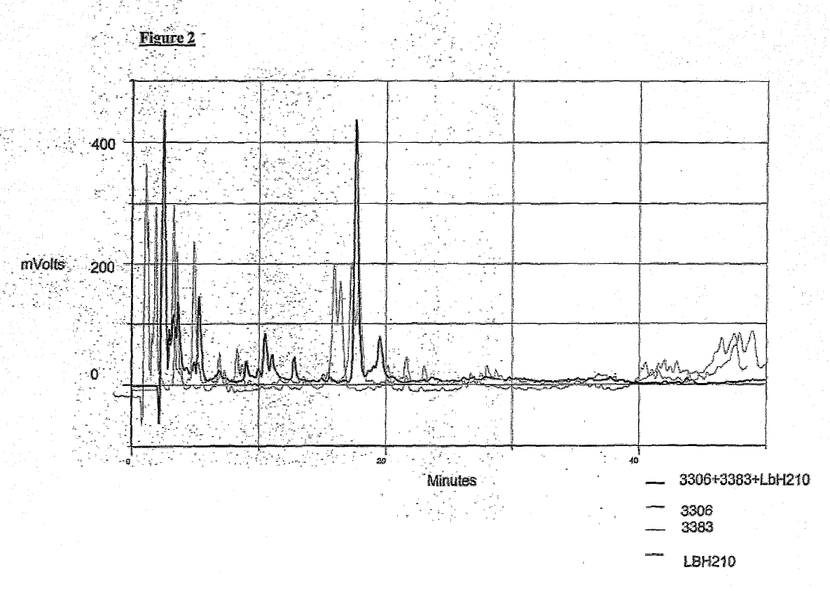

The invention will be better understood, and other purposes, details, characteristics and advantages of the invention will become clearer from the detailed explanatory description given below, of several embodiments of the invention, given purely as illustrative and non-limiting examples, referring to the attached drawings, FIGS. 1 and 2.

FIG. 1 shows a comparison of the effect of the use of a flavouring composition according to the invention with that of a conventional flavouring composition, on the production of aromatic products.

FIG. 2 shows the chromatograms obtained by HPLC from cheese samples manufactured and treated with the strains Brevibacterium 3306, Brevibacterium 3383, and the strain Lactobacillus helveticus LbH 210.

The invention relates to a composition for flavouring dairy products, in particular cheeses, comprising at least one combination of microorganisms, said combination comprising at least one microorganism of lytic type and at least one ripening agent.

The microorganisms of lytic type are for example the acidification or ripening lytic lactic acid bacteria, for example of the genera Lactococcus, Lactobacillus, Pediococcus and Leuconostoc of lytic type.

Advantageously, the microorganism of lytic type is of the genus Lactobacillus and preferably Lactobacillus helveticus. The genus Lactobacillus helveticus belongs to the lactic acid bacteria family. The bacteria belonging to this genus are Gram-positive bacteria, and generally catalase-negative, homofermentative or heterofermentative and producing lactate D or L from carbohydrates.

The bacteria Lactobacillus helveticus of lytic type are naturally present in milk and are widely used in the food-processing industry, and in particular in the cheese industry.

Preferably, said microorganism of lytic type is the strain Lactobacillus helveticus LbH 210 deposited at the CNCM on 20 Jan. 2006 under number CNCM I-3554.

The ripening agents are for example bacteria and in particular bacteria of the genera Arthrobacter, Corynebacterium, Lactobacillus, Lactococcus, Leuconostoc, Micrococcus, Pediococcus, Propionibacterium, Staphylococcus and Streptococcus. They can also be yeasts or moulds of the genera Candida or Debaryomyces, Geotrichum, Kluyveromyces, Rhodotorula, Saccharomyces or Penicillium.

Advantageously, said composition additionally contains at least one microorganism of the genera Arthrobacter, Brevibacterium, and advantageously Brevibacterium linens.

The genus Brevibacterium belongs to the family Coryneformes. The bacteria belonging to this genus are Gram+, heterofermentative bacteria.

In an advantageous embodiment, said ripening agent is the strain Brevibacterium 3306 deposited at the CNCM on 20 Jan. 2006 under number CNCM I-3556.

In another advantageous embodiment, said ripening agent is the strain Brevibacterium 3383 deposited at the CNCM on 20 Jan. 2006 under number CNCM I-3555.

In a particular embodiment, said microorganisms of lytic type and said ripening agents are in a mixture or in separate form.

Advantageously, said microorganisms of lytic type and said ripening agents are in the form of a mixture of lyophilized or frozen strains, or in the form of strains packed separately.

The strain Lactobacillus helveticus LbH 210 deposited at the CNCM on 20 Jan. 2006 under number CNCM I-3554 can also be added separately from the ripening agent.

A composition for flavouring dairy products according to the invention is used in a manner that is conventional for a person skilled in the art. In the case of manufacture of a dairy product, this will be carried out in a manner that is customary in this field, and in particular by fermentation of a dairy product by incorporating a ferment.

In particular, it is possible to mix, prior to inoculation of the medium to be fermented, in this case milk, the combination comprising at least one microorganism of lytic type and at least one ripening agent directly with the lactic acidification ferment or starter.

It is, however, possible for the flavouring composition comprising at least one microorganism of lytic type and at least one ripening agent and the lactic ferment to be incorporated separately or simultaneously in the medium to be fermented.

The microorganisms of lytic type and the ripening agents can be incorporated, simultaneously or separately, in dry, lyophilized or frozen form.

Another subject of the present invention is a method for flavouring a dairy product, in particular an uncooked pressed cheese, obtained by a method comprising the addition of a flavouring composition to the milk.

The dairy product contains milk of animal and/or vegetable origin.

As milk of animal origin, there can be mentioned cow's milk, goat's milk, sheep's milk, camel's milk, buffalo's milk or a combination thereof.

As milk of vegetable origin, there can be mentioned any fermentable substance of vegetable origin that can be used according to the invention in particular obtained from soya, rice, coconut or malt seeds.

In a particularly advantageous embodiment of the method, the microorganisms of lytic type and the ripening agents are added at a rate of 10⁶ to 10⁹ CFU per litre of milk, preferably approximately 10⁸ CFU per litre of milk. Typically the proportion of microorganisms of lytic type will be comprised between 30 and 70% relative to the total quantity of ripening agents. Thus, in the finished dairy product, the concentration of microorganisms of lytic type and of ripening agents is comprised between 10⁴ and 10⁸ CFU/g of dairy product, preferably approximately 10⁶ CFU/g of dairy product.

Advantageously, the dairy product is chosen from soft cheese, uncooked pressed cheese, cooked cheese, fromage frais, veined cheese, cheese spreads and “analogue cheese” (based on casein powder and/or whey proteins), enzyme modified cheese, cottage cheese, as well as yoghurt, ripened cream, milk drinks, a fermented milk, a dairy product retentate, a hydrolysate of vegetable proteins, for example soya, or a baby milk.

Even more advantageously, the dairy product is of the uncooked pressed cheese type, and preferably of the Gouda or Cheddar type.

Among the cheeses, there can be mentioned among others: Banon, Bleu d'Auvergne, Brie, Boulette d'Avesne, Caerphilly, Camembert, Cantal, Carré de l'Est, Chanco, Charource, Cheddar, Cheshire, Cotija, Coulommiers, Danbo, Dauphin, Double Gloucester, Edam, Emmenthal, Epoisse, Feta, Gorgonzola, Gouda, Jarisberg, Limberger, Livarot, Mimolette, Manchego, Maroilles, Monterey Jack, Mozzarella, Munster, Parmesan type, Pelardon, Pont-l'Evêque, Raclette, Red Leicester, Roquefort, Saint-Félicien, Saint-Marcellin, Saint-Nectaire, Saint-Paulin, Stilton, Tilsit, Tomme de Savoie, Vacherin Mont-d'Or and Vieux-Lille.

The following examples illustrate the invention though without limiting its scope.

Production of Cheese of the Cheddar Type:

A milk is reconstituted from skimmed milk powder incorporated at 930 g per 8.36 L of water. Then an aqueous solution of calcium chloride (Calciol® from Marshall at 500 g/L water) is added at a concentration of 0.35 mL/L of reconstituted milk.

The fermentation medium thus prepared is ready to be inoculated with acidification ferments (Choozit RA073® from Danisco) and either a flavouring composition according to the invention (containing a strain of Lactobacillus helveticus of lytic type LbH 210 together with two strains of the genus Brevibacterium 3383 and 3306) or a conventional flavouring composition (containing a strain of Lactobacillus of non-lytic type LB 67 together with two strains of the genus Brevibacterium 3383 and 3306).

Two 10 L vats of the Pierre Guerin® type are used in this way.

Then a quantity of cream is added (1000 g per vat) to obtain 33 grams of fats per litre of milk. The temperature is maintained at 32° C. and the stirring speed at 5 (for the 10 L vat of the Pierre Guerin® type).

The following are added simultaneously to the first vat: the lytic strain LbH 210 at a rate of 1×10⁸ CFU/L of fermentation medium, the strains 3383 and 3306 at a rate of 1×10⁸ CFU/L of fermentation medium and a conventional acidification ferment (Choozit RA073® (from Danisco) at a rate of 2×10⁹ CFU/L of fermentation medium.

The following are added simultaneously to the second vat: the non-lytic strain LB67 (deposited at the CNCM on 20 Jan. 2006 under number CNCM I-3553), at a rate of 2×10⁸ CFU/L of fermentation medium, the strains 3383 and 3306 at a rate of 1×10⁸ CFU/L of fermentation medium and a conventional acidification ferment (Choozit RA073® from Danisco) at a rate of 2×10⁹ CFU/L of fermentation medium.

Then in each of the vats, the mixture is ripened for 45 minutes under stirring (speed 5).

Rennet, having a concentration of 520 mg of chymosin per litre of rennet, is added to the mixture after ripening at a rate of 25 mL per 100 litres of milk. The pH is recorded during manufacture by means of a pH-meter. Coagulation takes 20 to 30 minutes.

After coagulation, the curd obtained is left to stand for 20 minutes and is then cut up. It is cut longitudinally and transversely. The curd and the whey are heated together from 32° C. to 42° C. and stirred at speed 5 for 120 minutes.

The whey that forms is removed continuously.

The curd from each of the vats is placed in two moulds of 14 cm diameter.

Then the curd from each mould is cut into equal pieces (4 to 8) approximately every 15 minutes in order to remove the whey, then returned.

Once the pH of each curd reaches 5.2, each curd is cut into small pieces (less than a centimetre in diameter) and 3% of NaCl by weight relative to the weight of the curd is added. Then each curd is mixed and moulded in a mould of 14 cm diameter and pressed successively at different pressures. The pressure exerted initially is 1.5 bar for 30 minutes then the pressure is increased every 30 minutes by 0.5 bar. Finally, a pressure of 3 bar is maintained overnight.

24 hours after inoculation, each moulded curd is weighed and wrapped impermeably in film under vacuum.

Then the cheeses are stored at 4° C. for approximately one week.

Next, cheese samples are submitted to an accelerated ripening procedure by slurrying, according to IBT standard method PM 4.0. This method is used in order to understand, in a time scale reduced to a week (instead of three months for conventional ripening), the biochemical mechanisms of ripening. Under these conditions the enzymatic processes involved in ripening are accelerated.

For this purpose, a 100 g sample of cheese (manufactured with a flavouring composition according to the invention or a conventional flavouring composition) is ground in 50 mL of water in the presence of 1 g of trisodium citrate and 300 mg of ascorbic acid. Each mixture is then placed under vacuum for a week at 30° C. in order to obtain a so-called “final suspension”.

Then tests are carried out on the final suspension obtained.

The first test consists of quantifying the presence of amino acids in each of the suspensions.

The results are given below in Table 1.

TABLE 1
Comparison of the quantity of amino acids obtained
with different ripening compositions:

| | Brevibacterium 3306 and | Brevibacterium 3306 and |
|---|---|---|
| | 3383 + Lactobacillus | 3383 + Lactobacillus |
| | helveticus LB 67 | helveticus LbH 210 |
| Quantity of amino | 10.2 | 10.9 |
| acids in mg/g of | | |
| final suspension | | |

In the presence of lytic microorganisms, a greater quantity of amino acids and therefore an enhanced flavour are obtained, as the amino acids are involved to a considerable extent in the development of the flavour of a dairy product.

Next, a sample of each of the final suspensions (obtained with a flavouring composition according to the invention or with a conventional flavouring composition) is analyzed by gas chromatography (GC) in order to determine the volatile compounds involved in the development of the flavour of a dairy product.

GC Protocol:

The following are introduced into a 10 g sealable bottle: 3 g sodium chloride Normapur at 99.5%, 5 g of sample for analysis (in the present case 5 g of “final” suspension), and 2 mL of ultrapure water.

Extraction is carried out with a device of the Headspace HS40XL® type from Perkin-Elmer. The heating time applied is 30 minutes, the temperature of the sample is 60° C., the temperature of the needle 80° C. and transfer temperature 100° C.

The separation and assay are carried out with a device of the GC Autosystem XL® type from Perkin-Elmer. The column used is of CP-SIL5CB (Varian®) type, of Wcot fused silica type, 30 m×0.32 mm (inside diameter).

The stationary phase consists of 100% dimethylpolysiloxane. The thickness of the film is 0.5 μm.

The carrier gas is helium at a flow rate of 1.6 mL/minute.

The following cycle is applied: 40° C. for 2 minutes then 10° C. up to 160° C., then 160° C. for 3 minutes.

The detector is a flame ionization type detector. The maximum temperature applied is 250° C.

The results are shown in FIG. 1.

The area of the chromatography peaks obtained is quantified in arbitrary units, which are shown along the x-axis of the graph in FIG. 1.

All of the following volatile compounds: i.e. diacetyl, 2-butanone, acetoin, DMDS, 1-octen-3-ol, DMTS and butyric acid volatile compound are present in significantly greater quantities when a composition incorporating a strain of Lactobacillus helveticus LbH 210 (lytic strain) combined with two strains of Brevibacterium is used than when a composition incorporating a strain of Lactobacillus helveticus LB 67 (non-lytic strain) combined with two strains of Brevibacterium is used. These results are all the more significant when twice the amount of non-lytic Lactobacillus helveticus as of lytic Lactobacillus helveticus has been incorporated in the samples for purposes of comparison.

Finally, various cheese samples were analyzed by reversed-phase liquid chromatography in order to determine the non-volatile compounds involved in the development of the flavour of a dairy product.

The various cheese samples were obtained from cheeses manufactured either with a flavouring composition comprising Lactobacillus helveticus LbH 210 (lytic strain) in the presence of Brevibacterium 3383 and 3306, or Lactobacillus helveticus LbH 210 (lytic strain) alone, or Brevibacterium 3383 alone, or Brevibacterium 3383 alone.

HPLC Protocol Used:

5 g of cheese from each sample, weighed beforehand, is ground in a Valentin in a 50 ml tube. Grinding each sample makes it possible to optimize extraction and to have a homogeneous sample.

Then 20 mL of citrate buffer pH3.0 is added to the ground cheese. Then the sample is homogenized in an Ultra-Turrax for 20 seconds. The operation is repeated until a homogeneous mixture is obtained. Then the tube is placed in an oven at 40° C. for 1 hour. After this stage, the fat is located on the surface. For better extraction, it is recommended to place the tubes in ice so that the fatty phase solidifies.

Once the fatty phase has been extracted, the sample is centrifuged at 3000 rpm for 35 minutes.

After centrifugation, the peptides are located in the supernatant.

For analysis by HPLC, 1 ml of supernatant must be removed, placed in a 1.5 ml Eppendorf® tube and centrifuged at 13000 rpm for 10 minutes.

After centrifugation, 1 ml of supernatant is removed using a filtration syringe and filtered through an Acrodisc Nylon 13-0.45 μm filter. The filtrate is recovered in an HPLC tube which is sealed. The sample is then ready to be analyzed by reverse phase HPLC using a Water/Acetonitrile gradient for a duration of 80 minutes, using a Phenomenex Jupiter® 10 μm C18 column, 300 Å 250×4.6 mm.

In addition, cheese samples were manufactured and treated in the same way as the samples mentioned previously, except that the microorganisms inoculated simultaneously with acidification ferment are either Brevibacterium 3306, or Brevibacterium 3383, or Lactobacillus helveticus LbH 210 strains.

The results are shown in FIG. 2 and Table 2 below.

TABLE 2
Comparison of the quantities of certain non-volatile
compounds involved in the flavour of cheese of
the Cheddar type in different samples:

| | Peak areas in arbitrary units | | | |
|---|---|---|---|---|
| | | | | 3306 + |
| | | | | 3383 + |
| | 3306 | 3383 | LbH210 | LbH210 |
| Elution at | 17,594,796 | 17,409,966 | 1,213,530 | 16,662,131 |
| approximately | (2 peaks) | (2 peaks) | (2 peaks) | (1 peak) |
| 17 minutes | | | | |
| Elution at | 272,259 | 312,993 | 451,919 | 4,650,971 |
| approximately | | | | |
| 18 minutes | | | | |
| Elution | 28,910,454 | 36,486,252 | 10,075,686 | 2,397,232 |
| between | | | | |
| 45 and 51 | | | | |
| minutes | | | | |

It was found that, compared with the use of the strains in isolation, with the use of the combination of the strains Brevibacterium 3306+Brevibacterium 3383+Lactobacillus helveticus LbH 210:
a considerable reduction of large, hydrophobic peptides (with elution time comprised between 45 and 51 minutes in FIG. 2) is achieved. These peptides generally impart bitterness to the dairy product and are to be avoided,
a significant quantity of small, hydrophilic peptides is obtained, the nature of which is modified (a single peak is obtained at approximately 17 minutes, the area of which is equivalent to the sum of the areas of the two peaks obtained during separate use of the ripening strains, in FIG. 2). These peptides generally impart flavours which are valued in dairy products (such as the taste of meat stock or savoury tastes for example) and
the appearance of small, hydrophilic peptides, which are not present when the strains are used in isolation (with elution time of approximately 18 minutes in FIG. 2) is obtained. These peptides belong to the range of peptides which generally impart flavours which are valued in dairy products and make it possible to vary or further mature the flavour obtained during ripening.

Although the invention has been described with reference to several particular embodiments, it is quite obvious that it is not in any way limited to these and that it includes all the technical equivalents of the means described as well as combinations thereof if the latter fall within the scope of the invention.

1. A Composition for flavouring dairy products comprising at least one combination of microorganisms, said combination comprising at least one microorganism of lytic type and at least one ripening agent.

2. A Composition according to claim 1, wherein the microorganism of lytic type is of the genus Lactococcus or Lactobacillus.

3. A Composition according to claim 1, wherein the microorganism of lytic type is of the species Lactobacillus helveticus.

4. A Composition according to claim 1, wherein the ripening agent is a microorganism of the genus Arthrobacter or Brevibacterium.

5. A Composition according to claim 1, wherein the ripening agent is of the species Brevibacterium linens.

6. A Composition according to claim 1, wherein said microorganism of lytic type is the strain Lactobacillus helveticus LbH 210 deposited at the CNCM on 20 Jan. 2006 under number CNCM I-3554.

7. A Composition according to claim 1, wherein said ripening agent is the strain Brevibacterium 3306 deposited at the CNCM on 20 Jan. 2006 under number CNCM I-3556.

8. A Composition according to claim 1, wherein said ripening agent is the strain Brevibacterium 3383 deposited at the CNCM on 20 Jan. 2006 under number CNCM I-3555.

9. A Composition according to claim 1, wherein said microorganisms of lytic type and said ripening agents are in a mixture.

10. A Composition according to claim 1, wherein said microorganisms of lytic type and said ripening agents are in the form of a mixture of lyophilized or frozen strains.

11. Strain Lactobacillus helveticus LbH 210 deposited at the CNCM on 20 Jan. 2006 under number CNCM I-3554.

12. A Method for flavouring a dairy product comprising the addition of a flavouring composition according to claim 1, in milk.

13. A Method according to claim 12, wherein the concentration of microorganisms of lytic type and of ripening agents is comprised between 10⁴ and 10⁸ CFU/g of dairy product.

14. A Method according to claim 12, wherein the microorganisms of lytic type and the ripening agents are added at a rate of 10⁶ to 10⁹ CFU per litre of milk.

15. A Method according to claim 12, wherein the milk is of animal origin.

16. (canceled)

17. (canceled)

18. A method according to claim 12, wherein the dairy product is selected from the group consisting of soft cheese, uncooked pressed cheese, cooked cheese, fromage frais, veined cheese, cheese spreads and “analogue cheese”, enzyme-modified cheese, cottage cheese, as well as yoghurt, ripened cream, milk drinks, a fermented milk, a dairy product retentate, a hydrolysate of vegetable proteins or a baby milk.

19. A method according to claim 12, wherein the dairy product is of the uncooked pressed cheese type.

20. A composition according to claim 1, wherein the microorganism of the lytic type is of the species Lactobacillus casei.

21. A kit comprising at least one microorganism of lytic type and at least one ripening agent in separate form.

22. A kit according to claim 21, wherein said microorganism of lytic type and said ripening agent are in the form of strains packed separately.
